(19) United States
(12) Patent Application Publication
(10) Pub. No.: US 2019/0328923 A1
(43) Pub. Date: Oct. 31, 2019

(54) ODOR NEUTRALIZER FOR AIR CONDITIONER

(71) Applicant: James Cunningham, Long Beach Township, NJ (US)

(72) Inventor: James Cunningham, Long Beach Township, NJ (US)

(21) Appl. No.: 15/966,547

(22) Filed: Apr. 30, 2018

(51) Int. Cl. A61L 9/12

(52) U.S. Cl. CPC A61L 9/12; F24F 1/0007

(57) ABSTRACT

An odor neutralizer, for placement on upward facing indoor air intake louvers of a “mini-split” or other ductless air conditioner, comprises a cylindrical container housing an evaporable odor neutralizing gel, and having rows of holes in the container covered by flexible strips that can be removed to expose the evaporable gel to air flowing into the intake louvers. The ends of the cylindrical container are closed by end caps shaped to raise the cylindrical container so that it does not block the louvers and so that it does not roll on the louvered surface of the air conditioner.

### FIELD OF THE INVENTION

This invention relates to odor neutralization, and more particularly to an odor neutralizer for eliminating odors caused by the presence of mold, smoke, bacteria, and other substances in air conditioners such as mini split air conditioners, packaged terminal air conditioners (P-Tacs) and hotel room air conditioning units.

### BACKGROUND OF THE INVENTION

A mini split air conditioner is a ductless air conditioner that comprises an outdoor unit with a compressor and a condenser, and a wall-mounted indoor unit having an evaporator and a blower. A refrigerant circulates from one unit to the other through a pair of conduits. The indoor units are easily installed, are very quiet and efficient, and allow for air conditioning, and also for heating, in rooms where the installation of ducts would be very difficult and expensive. Packaged terminal air conditioners and hotel room air conditioners are also ductless air conditioners, but differ from the mini split air conditioners in that the evaporator and blower are housed in the same unit in which the compressor and condenser are housed.

A problem with all of the above-mentioned ductless air conditioners is that odor-emitting substances such as mold, mildew, bacteria, smoke and the like tend to accumulate on the evaporator coil and on other components of the indoor unit. Odor emitting substances from pets, foods, and chemicals can also accumulate in the indoor units. The need for frequent cleaning and removal of these odor-emitting materials makes maintenance very difficult.

The indoor units of mini split air conditioners are very compact, and have very little interior space for the placement of an odor neutralizer. Packaged terminal air conditioners and hotel room air conditioners have similar problems.

### SUMMARY OF THE INVENTION

An object of this invention is to provide an odor neutralizer that can be placed directly on upward facing indoor air intake louvers of a ductless air conditioner so that vapor from the odor neutralizing composition contained within the neutralizer can flow into the blower whenever the air conditioner is running.

The neutralizer comprises a hollow, tubular container having an outer wall conforming to an imaginary cylinder extending along a cylinder axis. The container, which houses an odor neutralizing composition that evaporates at ambient temperatures, has two opposite ends spaced from each other in the direction of the cylinder axis. The tubular container is provided with an array of openings that extend through the outer wall, and the neutralizer is supplied with the openings covered by a removable strip that blocks the flow of vapor from the odor neutralizing composition through the openings.

The neutralizer includes a pair of end caps, one closing each of the opposite ends of the container. The end caps are shaped in such a way as to support the hollow tubular container in spaced relation to the air intake louvers of the air conditioner so that it does not block the flow of air through the louvers, and to prevent the neutralizer from rolling when set in place on the louvers.

In a preferred embodiment, each of the end caps includes a substantially polygonal portion having corners located radially outward relative to the imaginary cylinder to which the outer wall of the tubular container conforms. The polygonal portion engages an upward facing, louvered surface of the air conditioner and support the tubular container so that it is spaced above the louvers underneath it, allowing air to flow past the array of openings in the tubular container and though the louvers into the blower and evaporator section of the air conditioner.

Although the end caps preferably have a substantially polygonal portion, the polygonal shape is not absolutely essential. Each of the end caps can be formed, for example, with least one air conditioner-engaging surface facing radially outward relative to the cylinder axis. This surface should have two areas located radially outward from the imaginary cylinder, and spaced from each other in a direction in a first imaginary plane to which the cylinder axis is perpendicular and situated substantially in a second imaginary plane parallel to the cylinder axis, the portion of the air conditioner engaging surface between these two areas should be substantially limited to locations in the second imaginary plane and on the side of the second imaginary plane on which the cylinder axis is located. If the air-conditioner engaging surface is in contact with an upward facing louvered surface of an air conditioner the tubular container is supported in spaced relation to the louvers and the neutralizer is prevented from rolling.

Alternatively, the end caps of the neutralizer can be defined as having at least one surface facing radially outward relative to the axis of the imaginary cylinder. This surface includes two points located radially outward from the imaginary cylinder and circumferentially spaced from each other on a second imaginary cylinder radially larger than, and coaxial with the imaginary cylinder to which the outer wall of the tubular container conforms. A connecting portion of this surface extends from one of the two points to the other, and all of the connecting portion is located radially inward of the second imaginary cylinder. An odor neutralizer is poured and cured so that it becomes a gel in the form of a cylinder. The cylinder is then installed into the tubular container.

The tubular container is preferably formed with two rows of holes, the rows extending lengthwise on opposite sides of the container. Each of the rows of holes is covered by a removable strip. These two strips prevent evaporation of the gel until the odor neutralizer is ready for use. The strips are peeled away when then user wants to activate and install the odor neutralizer. By peeling the strips away, the user can activate and use the product without ever touching gel.

When the strips are removed and the odor neutralizer, is placed on the louvers of an air conditioner, the gel eliminates odors in the air conditioner unit, and in the space that is served by the unit. The gel formulation helps to prevent mold and bacteria growth in the evaporator coil and internal compartments of the unit.

### BRIEF DESCRIPTION OF THE DRAWINGS

FIG. 1 is a front elevational view of an odor neutralizer in accordance with the invention, partly broken away to reveal the gel within the tubular container, and showing one of the removable strips partially peeled away to expose the gel through a row of holes;

FIG. 2 is a rear elevational view of the odor neutralizer of FIG. 1;

FIG. 3 is a perspective view showing the odor neutralizer being placed on the louvers of an air conditioner unit;

FIG. 4 is a perspective view of the odor neutralizer showing a central axis of the tubular container and two imaginary reference planes;

FIG. 5 is an end elevational view of the odor neutralizer of FIG. 4, showing the central axis and one of the reference planes; and

FIG. 6 is an end elevational view of the odor neutralizer, showing a first imaginary cylinder to which the outer wall of the tubular container conforms, the central axis of the tubular container and a second imaginary cylinder used for reference in defining the shape of the end caps of the odor neutralizer.

### DETAILED DESCRIPTION OF THE PREFERRED EMBODIMENTS

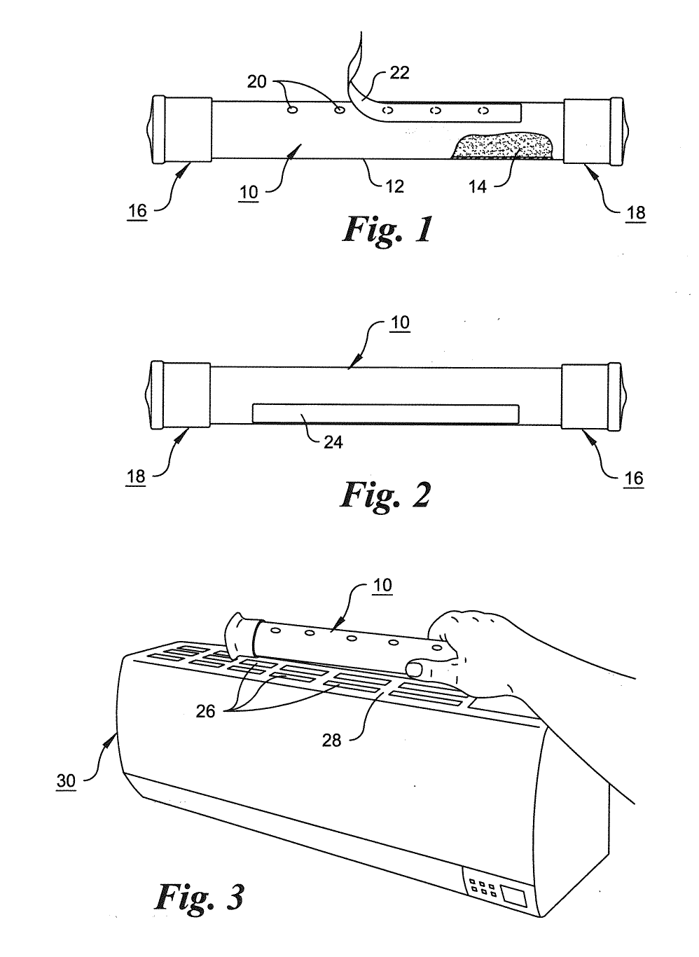

As shown in FIG. 1, the odor neutralizer includes a tube 10 having an outer wall 12 in the form of a circular cylinder and an interior space containing an odor neutralizing gel 14. The ends of the tube are closed by end caps 16 and 18, which receive the ends of the tube 10 and are glued to the external surface of the outer wall of the tube. A row of holes 20 extends lengthwise along the tube, and each hole 20 extends through the outer wall 12 so that vapor from the gel can pass through the holes and into an air conditioner on which the odor neutralizer is placed on louvers on the top of the air conditioner.

In the odor neutralizer as supplied, the row of holes 20 is covered by a removable flexible strip 22, which is held in place by an adhesive that allows the strip to be peeled away manually. The strip 22 is shown partially peeled away in FIG. 1.

A similar row of holes (not shown) extends lengthwise along the opposite side of the tube and is covered by a similar removable strip 24, shown in FIG. 2. The number of holes in each row is preferably five or six.

For use, the strips 22 and 24 are peeled away and the odor neutralizer is placed on the air intake louvers 26 on an upward facing part 28 of an air conditioning unit 30 as shown in FIG. 3. The air conditioning unit 30 in FIG. 3 is the evaporator and blower section of a mini-split air conditioner, i.e., the part of the air conditioner situated in the interior of a room. A compressor/condenser unit (not shown), associated with the evaporator and blower section, is situated on the exterior of the building. The odor neutralizer can also be utilized with other ductless air conditioning equipment, e.g., P-Tac air conditioners, hotel room air conditioners, and other air conditioning units that have intake louvers on an upward facing surface. In some cases, the louvers are disposed underneath a cover, and the odor neutralizer can be set in place by temporarily opening the cover, placing the odor neutralizer on the louvers, and replacing the cover, leaving the odor neutralizer in a space between the louvers and the cover.

If the odor neutralizing gel were contained in a cylinder having its ends closed by closures having the same transverse dimensions as the cylinder, the cylinder could tend to block the openings of the louvers and impair the efficiency of the air conditioner. If the cylinder is circular, or if its transverse cross-sectional shape includes a convex curve, it could also tend to roll, and could fall off the air conditioner. The end caps of the odor neutralizer in accordance with this invention address these problems by having end portions with surfaces that are located radially outward relative to the outer wall of the cylinder and that are shaped so that they do not tend to roll when engaged with a louvered upper surface of an air conditioner.

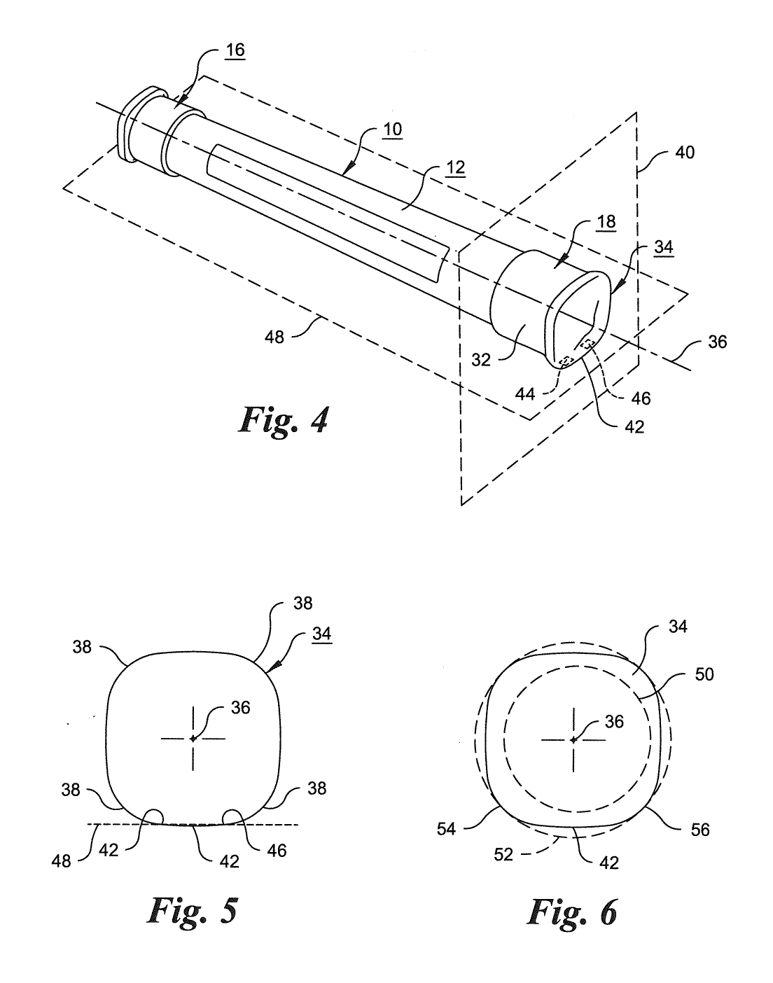

As shown in FIG. 4, end cap 18 includes a cylindrical part 32 that is radially larger than the outer wall 12 of tube 10 and a non-circular end portion 34 that is radially larger than the cylindrical part (with reference to longitudinal axis 36 of the cylindrical tube 10). As shown in FIG. 5, which depicts the shape of the end portion 34 as viewed along the axis 36, the preferred end portion 34 is substantially in the form of a rectangle, with rounded corners 38, connected by sides that are substantially straight, i.e., sides that are sufficiently straight that they prevent rolling of the odor neutralizer when placed on a horizontal surface.

As shown in FIG. 4, end portion 34 is intersected by an imaginary reference plane 40, to which axis 36 is perpendicular, and its bottom side surface 42 has two laterally spaced areas 44 and 46, which are located in an imaginary reference plane 48, which is parallel to axis 36 and perpendicular to reference plane 40. The portion of the bottom side between areas 44 and 46 can be straight or concave. It can also be slightly convex so that it projects a very short distance below reference plane 48 as shown in FIG. 5. In other words, the portion of the bottom side surface 42 between areas 44 and 46 is substantially limited to locations in imaginary reference plane 48 and on the side of said imaginary reference plane 48 on which the cylinder axis 36 is located. In any of these cases, the bottom side 42 will resist rolling of the odor neutralizer, when engaged with a louvered air intake on the top part of an air conditioning unit. The portion of the bottom side surface 42 is substantially in the imaginary reference plane 48 or on the side thereof on which the cylinder axis is located, provided that it does not protrude below reference plane 48 by an amount sufficient to permit rolling of the odor neutralizer off a planar surface disposed at a 20° angle relative to horizontal.

End cap 16 at the opposite end of the tube 10 is similar to end cap 18 and should be secured to the tube so that its edges are aligned with corresponding edges of end cap 18.

FIG. 6 illustrates an alternative way of defining the configuration of the end portion 34 of the end cap. A first imaginary cylinder 50 corresponds to the outer wall of the tubular container, and a second imaginary cylinder 52, coaxial with central axis 36, are used for reference in defining the shape of the end portion 34 of the end cap. The rounded corners of the end portion 34 extend radially outward relative to the first imaginary cylinder 50, and the second imaginary cylinder 52 is tangent to at least two radially outermost points 54 and 56 of the end portion 34. The bottom side surface 42 between the outermost points 54 and 56 is entirely inside imaginary cylinder 52. In other words, the bottom side surface 42 includes two points 54 and 56, located radially outward from imaginary cylinder 50. These points 54 and 56 are located, and circumferentially spaced from each other, on a second imaginary cylinder 52 radially larger than imaginary cylinder 50, and all of the portion of the bottom side surface 42 that connects points 54 and 56, is located radially inward of imaginary cylinder 52.

The frictional characteristics of the synthetic resin material of the end caps should be such that the odor neutralizer will not slide on a planar metal surface, having a frictional characteristic corresponding to the exterior surface of an air-conditioner housing and disposed at an angle up to 20° relative to a horizontal plane.

The odor neutralizer uses an odor neutralizer in the form of a gel that is pour into, and cured in, round tubes and then installed into the odor neutralizer tube 10. The tube 10 is preferably 1⅝ inches in diameter and 12 inches long. Each of the two rows of holes preferably consists of four or five ¼ inch holes, which covered with a removable peel-off strip of flexible white vinyl coated with an adhesive. A suitable material is the FASSON® LaserCode II 3483 XX vinyl material, available from Avery Dennison Corporation of Glendale, Calif. The peel-off strips prevent evaporation of the gel while it is on the shelf, and are removed when then user wants to activate and install the odor neutralizer on or in an air-conditioning unit. The user can activate and use the product without ever touching the solid gel odor neutralizer. The end caps not only serve to seal both ends of the odor neutralizer tube, but also prevent the odor neutralizer from rolling off the return louvers of the air conditioning unit. The end caps also elevate the odor neutralizer slightly above the air return louvers so that it does not impede air flow but is in the air flow path in order to feed the evaporating gel odor neutralizer into the air conditioning unit. The odor neutralizing gel eliminates odors in the air conditioning unit, and in the space that is served by the and prevents mold and bacteria growth in the evaporator coil and internal compartments of the air conditioning unit.

While the presently preferred embodiment of the odor neutralizer is as described above, various modifications can be made to the odor neutralizer as described. For example, although the preferred shape of the end portions of the end caps is square with rounded corners, other polygonal shapes can be used. For example, a triangular configuration can be used. However, with more sides, e.g., a pentagon or a hexagon, the odor neutralizer is more likely to roll. The end portions can also have irregular configurations provided that each of them has a substantially straight or concave surface that will prevent rolling of the odor neutralizer. The dimensions of the odor neutralizer, numbers of holes and hole configurations can also be varied.

What is claimed is:

1. An odor neutralizer for use with a ductless air conditioner having upward facing indoor air intake louvers, the neutralizer comprising:
a hollow container having an outer wall conforming to an imaginary cylinder extending along a cylinder axis, said container having two opposite ends spaced from each other in the direction of said cylinder axis;
an odor neutralizing composition that evaporates at ambient temperatures within said container; an array of openings extending through said outer wall of the container, said openings being covered by a removable strip that blocks the flow of vapor from said odor neutralizing composition through said openings; and
a pair of end caps, one of said end caps closing each of said opposite ends of the container, each of said end caps having at least one surface facing radially outward relative to said axis of the cylinder, said at least one surface including two areas located radially outward from said imaginary cylinder, said areas being spaced from each other in a direction in a first imaginary plane to which said cylinder axis is perpendicular and being situated substantially in a second imaginary plane parallel to said axis, the portion of said at least one surface between said two areas being substantially limited to locations in said second imaginary plane and on the side of said second imaginary plane on which said cylinder axis is located.

2. An odor neutralizer for use with a ductless air conditioner having upward facing indoor air intake louvers, the neutralizer comprising:
a hollow container having an outer wall conforming to a first imaginary cylinder extending along a cylinder axis, said container having two opposite ends spaced from each other in the direction of said cylinder axis;
an odor neutralizing composition that evaporates at ambient temperatures within said container;
an array of openings extending through said outer wall of the container, said openings being covered by a removable strip that blocks the flow of vapor from said odor neutralizing composition through said openings; and
a pair of end caps, one of said end caps closing each of said opposite ends of the container, each of said end caps having at least one surface facing radially outward relative to said axis of said imaginary cylinder, said at least one surface including two points located radially outward from said first imaginary cylinder, said points being located, and circumferentially spaced from each other, on a second imaginary cylinder radially larger than said first imaginary cylinder, and a connecting portion of said at least one surface extending from one of said points to the other, all of said connecting portion being located radially inward of said second imaginary cylinder.

3. The odor neutralizer according to claim 2, in which said second imaginary cylinder is coaxial with said first imaginary cylinder

4. An odor neutralizer for use with a ductless air conditioner having upward facing indoor air intake louvers facing air intake louvers, the neutralizer comprising:
a hollow container having an outer wall conforming to an imaginary cylinder extending along a cylinder axis, said container having two opposite ends spaced from each other in the direction of said cylinder axis;
an odor neutralizing composition that evaporates at ambient temperatures within said container;
an array of openings extending through said outer wall of the container, said openings being covered by a removable strip that blocks the flow of vapor from said odor neutralizing composition through said openings; and
a pair of end caps, one of said end caps closing each of said opposite ends of the container, each of said end caps including a substantially polygonal portion having corners located radially outward relative to said imaginary cylinder.

5. The odor neutralizer according to claim 4, in which the polygonal portion of each of said end caps is in the form of a square with rounded corners.
